(19) United States
(12) Patent Application Publication
(10) Pub. No.: US 2017/0007410 A1
(43) Pub. Date: Jan. 12, 2017

(54) IMPLANTABLE ENCAPSULATED PROSTHETIC JOINT MODULE

(71) Applicant: John H. Sojka, Pasadena, CA (US)

(72) Inventor: John H. Sojka, Pasadena, CA (US)

(21) Appl. No.: 15/184,984

(22) Filed: Jun. 16, 2016

Related U.S. Application Data
(60) Provisional application No. 62/190,244, filed on Jul. 8, 2015.

(51) Int. Cl. A61F 2/32

(52) U.S. Cl. CPC A61F 2/32; A61F 2002/30584

(57) ABSTRACT

An implantable prosthesis component for a joint prosthesis includes a flexible wall, a proximal side comprising a rigid portion for connecting to a first bone at a joint; and a distal side comprising a rigid portion for connecting to a second bone at the joint. The flexible wall has an inverted spherical shape and defines an inner cavity of the implantable prosthesis component. The inner cavity is filled with a fluid. The flexible wall is deformable such that relative movement between the first and second bone causes deformation of the implantable prosthesis component.

### CROSS-REFERENCE TO RELATED APPLICATION(S)

This application claims the benefit of U.S. Provisional Patent Application Ser. No. 62/190,244, filed Jul. 8, 2015, the content of which is hereby incorporated by reference in its entirety.

### BACKGROUND

Joint prosthetics use various plasticized and or metalloid components to create contacting articulating surfaces to mimic the native joints they have replaced. The contacting surfaces slide past each other similar to the interaction of adjacent bones at a joint, for example, the interaction between the femoral head of the femur and the acetabulum of the pelvis. The frictional environments between these contacting surfaces have been extensively studied in hopes of determining which combination of materials provide the lowest friction state and therefore the most optimal longevity of the implanted components. Despite advances in composite materials, wear between the contiguous contacting surfaces persists and limits joint prosthetics to an average of fifteen years.

Attempts have been made to increase joint prosthesis longevity by providing designs that cushion joints and/or create lower-friction environments, for example, U.S. Pat. No. 8,979,938 to Linares, U.S. Pat. No. 7,175,666 to Yao, U.S. Pat. No. 5,389,107 to Nasser, U.S. Pat. No. 7,186,364 to King and 2004/0068322 to Ferree. These designs, however, fail to address another problem inherent to environments that continue to have contacting articulating surfaces. The contact and relative motion between the articulating surfaces creates unwanted erosion and debris production, leading to the creation of micro-particles, such as metal ions, that are released into circulation potentiating the risk of systemic disease or reaction. Reaction to these micro-particles are also known to be the major cause of osteolysis at the bone-implant interface. This osteolysis leads to aseptic loosening of the implant, which one of the most common indications for revision surgery. Scott J. MacInnes et al., “Risk Factors for Aseptic Loosening Following Total Hip Arthroplasty,” Recent Advances in Arthroplasty 275-294 (2012), Dr. Samo Fokter (Ed.), ISBN: 978-953-307-990-5, InTech, Available from: http://www.intechopen.com/books/recent-advances-in-arthroplasty/risk-factors-for-aseptic-loosening-following-total-hip-arthroplasty. Thus, previous lower-friction designs may decrease but not resolve the problems associated with joint prostheses.

Further, various current prosthetic designs do not wear evenly over their entire articulating surfaces. Rather, wear is found to occur within a specific zone of the articulating surface, which correlates to the uneven distribution of surface contact pressures. Contact pressures are found to be highest where the magnitude of the loading force vector is at a maximum and then decrease precipitously in all directions away from that point. Since the wear of prosthetic components are increased in these relatively small areas of concentrated contact pressures, the ideal prosthesis would be designed to distribute forces transferred through the joint evenly over the largest possible surface area, whereby exponentially lowering contact pressures and resultant wear rates. All current and proposed prosthetic joint designs are inherently unequipped to address this concern because they are all based on the same fundamental joint design. Only a truly novel prosthetic joint design will be able to address the aforementioned concern.

In addition to the above concerns, prosthetic joints also carry the risk of dislocation throughout the life of the prosthetic. A variety of methods have been used to minimize this risk. These methods include maximizing implant size and optimizing angles of implantation. However, these methods have not eliminated this risk altogether, and many patients with such prostheses must live with a limited range of motion. For example, patients with a hip prosthesis are typically instructed not to engage in strenuous activities or high impact activities in order to reduce the risk of dislocation. Dislocation can be unpleasant for patients, and require a long period of recovery and further restrictions on movement. Complications of dislocation include need for re-operation with or without revision surgery, sudden acute severe pain, functional impairment, soft-tissue damage, disassociation of modular components, and devastation to the patients' confidence in their hip replacement and/or surgeon. These complications and others, for example, are discussed in “Impingement in Total Hip Replacement: Mechanisms and Consequences,” Thomas D. Brown, Ph.D. et al., Curr. Orthop. 2008 Dec. 1; 22(6): 376-391, and “Risk of dislocation using large- vs. small-diameter femoral heads in total hip arthroplasty,” Johannes F. Plate et al., BMC Research Notes 2012, 5:553, both of which are attached as an Appendix to the present application.

With an increasingly aging population, there is great need for prosthetics with improved longevity. The ultimate goal being one joint replacement per lifetime, mainly to avoid the hazards of revision surgery, which is associated with longer hospital stays, poorer functional outcomes, and higher rates of in-hospital mortality. Scott J. MacInnes et al., “Risk Factors for Aseptic Loosening Following Total Hip Arthroplasty,” Recent Advances in Arthroplasty 275-294 (2012), Dr. Samo Fokter (Ed.), ISBN: 978-953-307-990-5, InTech, Available from: http://www.intechopen.com/books/recent-advances-in-arthroplasty/risk-factors-for-aseptic-loosening-following-total-hip-arthroplasty.

Therefore, there is a need for a prosthesis that avoids the need for contacting articulating surfaces, thus reducing friction and reducing the risk of micro-particles' release. There is also a need for a prosthesis that can transfer forces evenly over the largest possible surface area to lower contact pressures, reduce wear rates, and increase longevity. There is also a need for a prosthesis that allows for a patient to have an increased range of motion without or with a substantially decreased risk of dislocation. There is also a need for a prosthesis whose parts of those enduring wear can undergo easier replacement, thereby decreasing the need for extensive revision surgeries.

### SUMMARY

An implantable prosthesis component for a joint prosthesis includes a flexible wall, a proximal side including a portion for connecting to a first bone at a joint; and a distal side comprising a portion for connecting to the second bone at the joint. The flexible wall has an inverted shape and defines an inner cavity of the implantable prosthesis component. The inner cavity is filled with a fluid. The flexible wall is deformable such that relative movement between the first and second bone causes deformation of the implantable prosthesis component.

### BRIEF DESCRIPTION OF THE DRAWINGS

Further features and advantages of the invention will become apparent from and will be best understood by reference to the following detailed description reviewed in conjunction with the description of embodiments by means of the accompanying drawings. In the drawings:

FIG. 1 is an exploded perspective view of an implantable modular component as part of a hip prosthesis according to an illustrative embodiment of the invention.

FIG. 2 is a cross-sectional view of the hip prosthesis according to FIG. 1.

FIG. 3 is a cross-sectional view of the hip prosthesis according to FIG. 1 after implantation into a patient.

### DETAILED DESCRIPTION

Embodiments of the present invention disclose an implantable modular component for an artificial joint prosthesis. The implantable component may be a multi-part modular system that, when combined, forms a single unit that will serve to improve articulation of the joint prosthesis. The implantable component includes an articulating interface that is encapsulated and fluid-filled to reduce or prevent contact between surfaces during movement of the joint, thus reducing friction, wear, and the potential for micro-particle creation. This fluid filled capsule also causes forces transferred through the joint to evenly distribute over the entire encapsulated surface area, thus minimalizing potential for creating areas of pressure concentration and associated prosthetic components wear. In addition, because the capsule secures the two articulating surfaces within the modular system, the potential for dislocation is significantly reduced. The joint prosthesis incorporating the articulating interface may be adapted for use with various joints throughout the body, including but not limited to, the hip joints, knee joints, ankle joints, metatarsalphalangeal joints, interphalangeal joints, metacarpalphalangeal joints, elbow joints, shoulder joints, and vertebral joints.

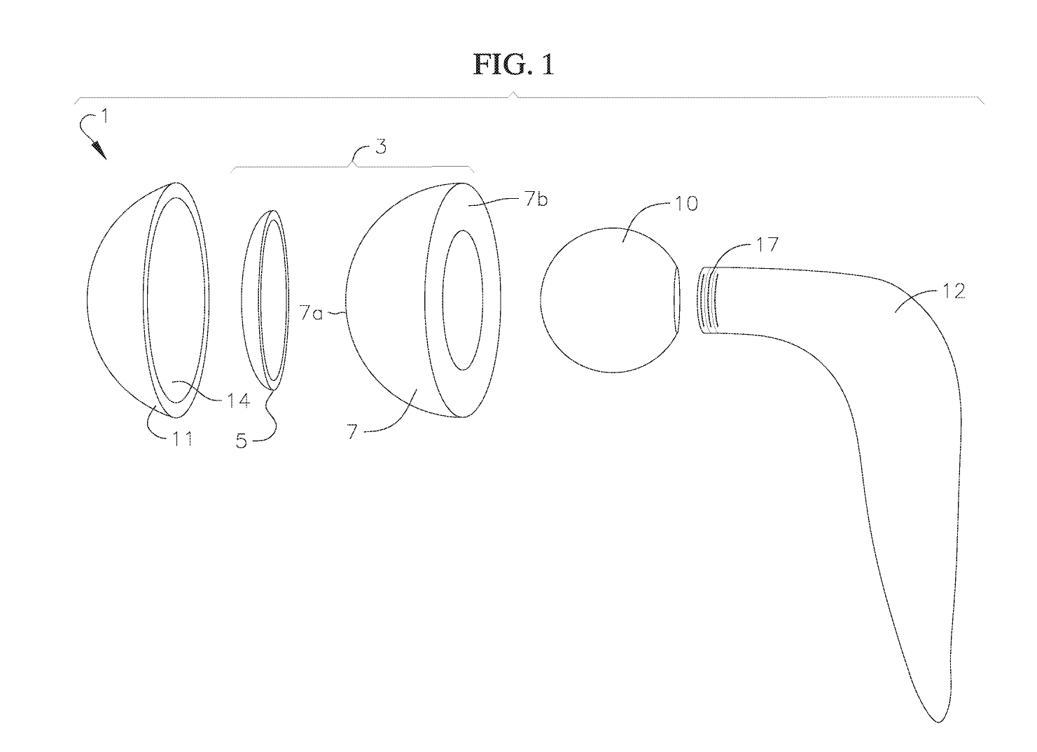

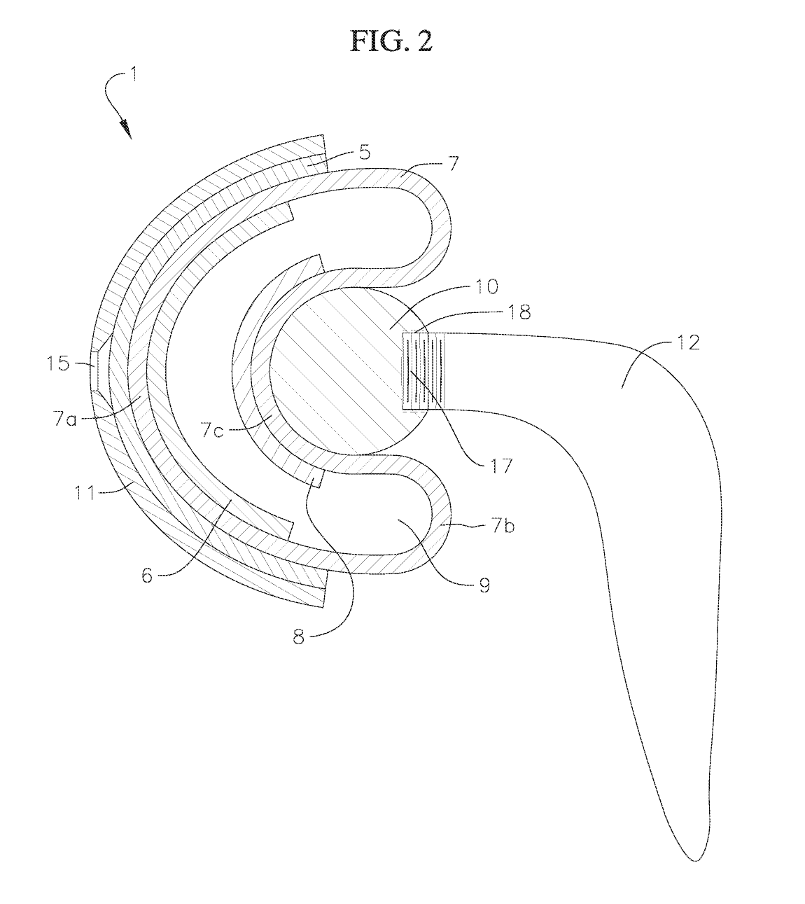

FIG. 1 shows an exploded perspective view of an implantable modular component 3 as part of a hip prosthesis 1, according to an illustrative embodiment of the invention. FIG. 2 shows a cross-sectional view of the same embodiment.

In the illustrative embodiment, the implantable component 3 is modular and includes an outer cap 5, a capsule 7, an inner proximal cap 6 and an inner distal cap 8. The outer cap 5 can be made of a metal, such as titanium, stainless steel, cobalt chrome, titanium alloys, nickel alloys, or other biocompatible metals. The outer cap 5 can also be made out of a plastic material, such as a polyethylene composite plastic material, or ceramic based material. The outer cap 5 may have a concave shape that is complementary to an outer surface shape of the capsule 7. The capsule 7 can have an inverted spherical shape, where a portion 7c of the sphere is inverted inside itself creating the hollow spherical segment shape of the capsule 7. The capsule 7 may also have other inverted shapes, such as an inverted ellipsoidal shape. The distance between a proximal end 7a of the capsule 7 and a proximal end of the inverted portion of the capsule 7 may be in the range of 2 mm to 20 mm, or more preferably 5 mm to 20 mm. The external diameter of the capsule 7 is dependent on both the size of the patient's femoral head and acetabulum, and will typically be within the range of 30 mm to 90 mm, or preferably 30 mm to 60 mm. The capsule 7 is made of a biocompatible flexible material, such as a silicone rubber. An inner cavity 9 of the capsule 7 is filled with a fluid, such as synovial fluid, air, or saline, as shown in FIG. 2. The inner cavity 9 is filled with a sufficient fluidic volume so that the wall at the inverted portion 7b will not touch the wall at a proximal end 7a of the capsule 7 at temperatures and pressures to which the hip joint is typically subjected or, in other embodiments, at temperatures and pressures to which a joint having the prosthesis is installed is subjected.

The outer cap 5 and the capsule 7 may be rigidly connected such that they do not move relative to one another. For example, the outer cap 5 and the capsule 7 may be connected by one or more screws, such as locking screws to prevent relative movement. Other attachment means such as a latching mechanism can also be used. The outer cap 5 is connected to the capsule 7 such that its concave shape is connected to the corresponding complementary outer surface shape of the capsule 7. The outer cap 5 can be connected to the proximal end 7a of the capsule 7 either directly or indirectly by securely sandwiching proximal end 7a of the capsule 7 between the outer cap 5 and the inner proximal cap 6, which have been rigidly connected to each other.

As shown in FIG. 2, both the inner proximal cap 6 and the inner distal cap 8 may be attached to the inner surface of the capsule 7. The inner proximal cap 6 can be located at the proximal end 7a of the capsule 7 and the inner distal cap 8 can be located at a distal end 7b of the capsule 7. The inner proximal cap 6 and the inner distal cap 8 can be made of a metal, such as titanium, stainless steel, cobalt chrome, titanium alloys, nickel alloys, or other biocompatible metals. The inner proximal cap 6 and the inner distal cap 8 can also be made out of a plastic material, such as a highly cross-linked polyethylene composite, or a ceramic based material. The inner proximal cap 6 and the inner distal cap 8 are generally shaped to correspond to the shape of the inner surface of the capsule 7, and thus may have the shape of a segment of a sphere or ellipsoid, for example, an a diameter corresponding to the external diameter of the capsule 7. The inner proximal cap 6 and the inner distal cap 8 may have a thickness in the range of 2 mm to 8 mm, or preferably 3 mm to 6 mm. The inner proximal cap 6 can be rigidly connected or interlocked to the outer cap 5 and the capsule 7, for example through one or more locking screws which also secures the outer cap 5 and the capsule 7, which can lock and seal the capsule 7 between the inner proximal cap 6 and the outer cap 5 at the proximal end 7a. The seal is air-tight to prevent leakage of the capsule 7. In embodiments in which the capsule contains a specific fluid, such as synovial fluid or saline, the capsule should be sufficiently tight to avoid leakage of that fluid. If any alternative fluid is used, the seal should be sufficient to prevent leakage of fluid into or out of the capsule.

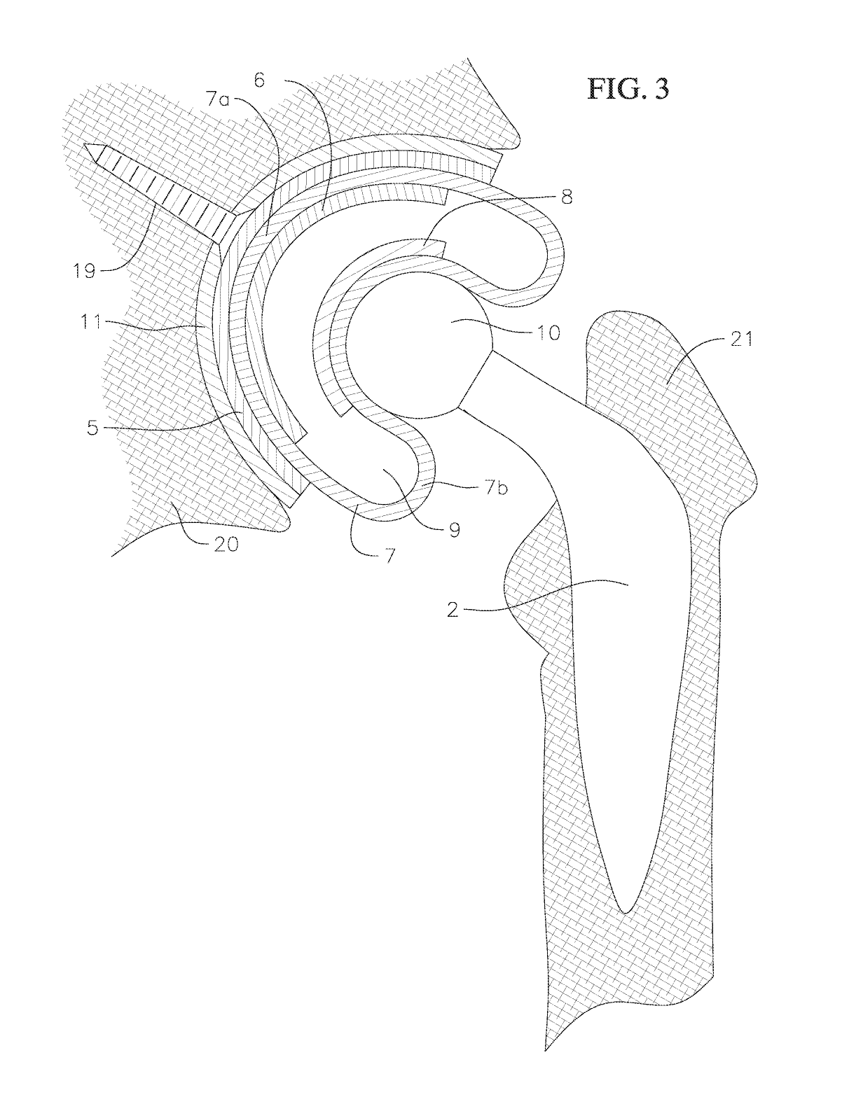

The implantable component 3 is part of the hip prosthesis 1, which also includes a head 10, an acetabular cup 11 and a femoral implant 12. The acetabular cup 11 may be sized to fit within the acetabulum 20 (shown in FIG. 3). The acetabulum may be reamed and unwanted tissue may be removed, after which the acetabular cup 11 may then be seated within the prepared acetabulum 20. The acetabular cup 11 is generally semispherical in shape and provided with a receiving cavity 14 to receive the implantable component 3. The acetabular cup 11 has an attachment portion (e.g., a hole) 15 for attaching the acetabular cup 11 to the acetabulum 20. The attachment portion 15 may be a threaded or bored for receiving a screw 19 to be anchored to the acetabulum 20, as shown in FIGS. 2 and 3. While there is one attachment portion 15 in this embodiment, other embodiments may include multiple attachment portions for attaching the acetabular cup 11 to the acetabulum 20. Each of these attachment portions may be threaded or bored for receiving a screw 19 to be anchored to the acetabulum 20. While all of these attachment portions may be used to attach the acetabular cup 11 to the acetabulum 20, in some embodiments, only some of these various attachment portions may be used in order to allow for variable screw placement (e.g., to allow a surgeon to determine which attachment portion he/she will use intraoperatively). Various attachment portions can be beneficial during surgery due to the variable aterial supply to the hip of patients. Having various attachment portions can provide the surgeon with options so as to avoid placing a screw or other attachment means through a major blood vessel.

In some embodiments, the acetabular cup 11 may also have pores to permit bone ingrowth to increase security of fixation to the prepared acetabulum 20. These pores may be on the surface of the acetabular cup 11 that contacts the acetabulum 20 and may be microscopic in size.

The acetabular cup 11 can be locked or rigidly connected to the proximal end 7a of the capsule 7, and the proximal end 7a of the capsule 7 can be either directly or indirectly locked between the inner proximal cap 6 and the outer cap 5, thus forming a rigid connection between the acetabular cup 11 and the flexible capsule 7.

The head 10 is generally spherical in shape and is attached to or accepts the corresponding portion of the femoral implant 12. Alternatively, the head 10 may be ellipsoidal in shape. The femoral implant 12 may have an protruding portion 17 that corresponds to a complementary accepting portion 18 in the head 10. This attachment may be loosely associated or rigidly fixed. For example, the attachment portion 17 may have a threaded portion that corresponds to grooves at portion 18 in the head 10. In some embodiments, the attachment portion can include a projection or groove that has an interference fit with the complementary attachment portion, such as via a latching or other internal locking mechanism. The femoral implant 12 may be a known or standard industry femoral implant known to persons in the art, as long as it is compatible with the head 10. The head 10 can be locked or rigidly connected, such as via a locking screw, at the inverted portion 7b of the capsule 7 that is between the inner distal cap 8 and the head 10, forming a rigid connection and seal between the head 10 and the flexible capsule 7. The seal is air-tight to prevent air from leaking out of the capsule 7. Again, if an alternative fluid is used, the seal should be sufficient to retain that fluid. In further embodiments, a distal outer cap may reside between the capsule 7 and the head 10 and the head 10 may be attached or locked to the capsule 7 through the distal outer cap.

The head 10 can be made of a metal, such as titanium, stainless steel, cobalt chrome, titanium alloys, nickel alloys, or other biocompatible metals. The head 10 can be made of plastic material, such as polyethylene, or a ceramic based material. The size of the head 10 will be dependent on the size of the capsule selected for a given patient, and may generally range between 20 mm to 50 mm at its outermost diameter.

The acetabular cup 11 can be spherical or ellipsoidal in shape and can be made of a metal, such as titanium, stainless steel, cobalt chrome, titanium alloys, nickel alloys, or other biocompatible metals. The external diameter of the acetabular cup 11 is dependent on the size and shape of the patient's femoral head, and will typically be within the range of 40 mm to 100 mm, or preferably 40 mm to 90 mm. The thickness of the acetabular cup 11 can be in the range of 2 mm to 6 mm, or prefereably 3 mm to 5 mm, or more preferably about 4 mm.

FIG. 3 is a cross-sectional view of the hip prosthesis according to FIG. 1 after implantation into a patient.

For implantation, the acetabulum 20 may be reamed to accurately fit the acetabular cup 11. The acetabular cup 11 may then be installed and attached to acetabulum 20 via the screw 19. Although a single screw is used in this embodiment, more than one screw or one or more other attachment means may be used to securely fasten the acetabular cup 11 to the acetabulum 20, for example, through multiple attachment portions. The acetabulum 20 may be pre-drilled to receive the screw 19 or the screw 19 may be self-tapping.

The femoral head is removed from the femur 21 along with a sufficient amount of extraneous bony tissue to allow installation and proper fitting of the femoral implant 12. In many instances, the patient receiving the surgery will have a damaged or osteoporotic femoral head, and such bone tissue will need to be removed to install the femoral implant 12. If a standard or known femoral implant is used, this installation of the femoral implant 12 may occur according to known procedures in the industry.

The implantable component 3, including the outer cap 6, the inner proximal cap 6, the capsule 7, and the inner distal cap 8, may be preassembled with the head 10 as a unit prior to implantation. The preassembled implantable component 3 and head 10 can then be fastened to the implanted acetabular cup 11, such as by a latch or screw mechanism. If problems with the implantable component 3 arise, the implantable component 3 can be removed and replaced without having to remove the acetabular cup 11 or the femoral implant 12, thus facilitating the replacement process and reducing the recovery time of the patient.

In use, because the capsule 7 is attached to the acetabulum 20 and the femur 21, relative movement of those two bones causes deformation of the capsule 7. Because the capsule is air-tight and contains a sufficient fluidic volume to avoid collapse under typical use by a patient, the inner cavity will maintain a constant encapsulated space between the inverted portion 7c and the rest of the capsule 7. The encapsulated space eliminates or substantially reduces the wear and tear due to friction that exists at the contacting surfaces of the prior designs. It also eliminates or substantially reduces the potential for micro-particle release, which also leads to prosthesis failure and can cause systemic disease. The encapsulated cushion of fluid also provides the added benefit of dampening impact at the joint, which may reduce the rate of microfracturing of other prosthesis components. The encapsulated fluid also causes forces traveling through the joint to disperse evenly across the entire encapsulated surface, eliminating the zones of pressure concentration that plague current designs. It also eliminates or substantially reduces the risk of dislocation due to the fact it secures the two articulating surfaces normally at risk of separation in current prosthetic designs. Further, it is possible this modular system may be surgically removed and replaced without the need to remove and replace the acetabular cup 11 and femoral implant 12.

Various modifications of the previous embodiments are conceivable. The ideal capsular material would have both excellent tensile strength and flexibility to reduce the risk of capsule failure during impact movements at the joint. Though silicone rubber is proposed here, many elastomers do exist that may fit this need. It is also possible there are some unrealized elastomers or combination of elastomers that may best fulfill these desirable qualities. Another possibility is to chemically and/or molecularly alter silicone rubber through various means until its physical properties are optimized for the purposes of this modular system. One possible well-known method, is through the utilization of different curing methods wherein the material is exposed various combinations of chemical, temperature, and pressure environments. Another method would be to combine the silicone rubber material with one or more additives for the purposes creating some form of a polymeric composite biomaterial with desired physical properties. Yet other possibilities considered here is to line or impregnate the silicone rubber capsule with a plasticized, fibrous, metallic, or other formidable mesh inlay. The capsule may also be formed by a material that is not completely air-tight, which may allow some lubricating fluid flow though it into the joint capsule, but is sufficient for maintaining an adequate joint space, such as a metal mesh alone.

In addition, in embodiments of the invention, the volume of fluid used in the capsule may vary, for example, based on the physical activities of the patient. A very active patient who engages in impact sports may require additional fluid volume to withstand the impact of the patient's activities, whereas an older or less active patient may not require the same amount or fluidic volume. In addition, the weight of the patient may also effect the volume of fluid needed, with a heavier patient potentially requiring a larger volume than a lighter patient. As the pressure increased in active or heavier patients, the thickness of the capsule 7 may also need to be increased to handle such loads.

The above invention is not limited to the hip joint, and may be used in various other joints of the body, such as knee joints, interphalangeal joints, elbow joints or shoulder joints, with dimensions of the components and fluidic volume of the capsule 7 modified to fit the requirements of those particular joints.

While the present invention has been described in connection with certain exemplary embodiments, it is to be understood that the invention is not limited to the disclosed embodiments, but is instead intended to cover various modifications and equivalent arrangements included within the spirit and scope of the of the appended claims, and equivalents thereof.

What is claimed is:

1. An implantable prosthesis component for a joint prosthesis, comprising:
a flexible wall having an inverted shape, the flexible wall defining an inner cavity of the implantable prosthesis component that is filled with a fluid;
a proximal side comprising a portion for connecting to a first bone at a joint; and
a distal side comprising a portion for connecting to a second bone at the joint;
wherein the flexible wall is deformable such that relative movement between the first and second bone causes deformation of the flexible wall.

2. The implantable prosthesis component on claim 1, wherein the portion of the proximal side comprises a metal cap that attaches to an outer surface of the flexible wall at a proximal side of the implantable prosthesis component.

3. The implantable prosthesis component on claim 2, wherein the portion of the proximal side further comprises an inner cap that attaches to an inner surface of the flexible wall at a proximal side of the implantable prosthesis component.

4. The implantable prosthesis component on claim 2, wherein the metal cap and the outer surface of the flexible wall are rigidly connected by an airtight seal that prevents fluid from leaking out of the inner cavity.

5. The implantable prosthesis component on claim 1, wherein the portion of the distal side comprises an inner cap that attaches to an inner surface of the flexible wall at an inverted portion of the implantable prosthesis component.

6. The implantable prosthesis component on claim 5, wherein the inner cap at the distal side and the inner surface of the flexible wall are rigidly connected by an airtight seal that prevents fluid from leaking out of the inner cavity.

7. The implantable prosthesis component on claim 1, wherein the fluid is air.
